(19) United States
(12) Patent Application Publication
(10) Pub. No.: US 2022/0193163 A1
(43) Pub. Date: Jun. 23, 2022

(54) COMPOSITION COMPRISING ACTINIDIA POLYGAMA EXTRACT FOR ALLEVIATING SKIN DAMAGE OR MOISTURIZING SKIN

(71) Applicant: G&P BIOSCIENCE CO., LTD., Seoul (KR)

(72) Inventors: Su Jin PARK, Seoul (KR); Seong-Hyun HO, Seoul (KR)

(73) Assignee: G&P BIOSCIENCE CO., LTD., Seoul (KR)

(21) Appl. No.: 17/605,483

(22) PCT Filed: Apr. 24, 2020
(86) PCT No.: PCT/KR2020/005464

§ 371 Date: Jan. 25, 2022

(30) Foreign Application Priority Data
Apr. 24, 2019 (KR) 10-2019-0047944
Nov. 18, 2019 (KR) 10-2019-0147340
Apr. 24, 2020 (KR) 10-2020-0049686

(51) Int. Cl. A61K 36/18; A23L 33/105; A61P 17/16

(52) U.S. Cl. CPC A61K 36/18; A61P 17/16; A23L 33/105

(57) ABSTRACT

The present disclosure relates to a composition for alleviating ultraviolet ray-induced skin damage or moisturizing skin, more particularly, to a composition containing an Actinidia polygama extract as an active ingredient. The composition can alleviate the reduction of skin moisture caused by ultraviolet ray-induced skin barrier damage and the resultant skin dryness, reduced skin elasticity and increased skin roughness and, as such, can be utilized as a food composition for alleviating ultraviolet ray-induced skin damage or moisturizing skin and, furthermore, as a health functional food or pharmaceutical composition, an animal feed composition, a pharmaceutical composition for animals or a cosmetic composition.

### TECHNICAL FIELD

The present disclosure relates to a composition for alleviating ultraviolet ray-induced skin damage or moisturizing skin, more particularly, to a composition for alleviating ultraviolet ray-induced skin damage or moisturizing skin, a food composition for alleviating ultraviolet ray-induced skin damage or moisturizing skin, a health functional food for alleviating ultraviolet ray-induced skin damage or moisturizing skin, an animal feed composition for treating ultraviolet ray-induced skin damage, an oral pharmaceutical composition for treating ultraviolet ray-induced skin damage, an oral pharmaceutical composition for animals for treating ultraviolet ray-induced skin damage, or a cosmetic composition for alleviating ultraviolet ray-induced skin damage or moisturizing skin, which contains a gaedarae, extract as an active ingredient. It also relates to a method for treating ultraviolet ray-induced skin damage by administering the composition and a novel use of the gaedarae extract for preparing a medication or a medication for animals for treating ultraviolet ray-induced skin damage.

### BACKGROUND ART

Skin is the largest tissue which covers the body. It defends the body against external stimuli and bacterial invasion and protects the body through body temperature regulation, sensation, waste excretion, etc.

Skin also ages like other body organs. Skin aging is classified into intrinsic aging resulting from decline in human body functions and alteration in physiological functions such as hormones, and extrinsic aging caused by various environmental factors including ultraviolet ray. Ultraviolet ray-induced photoaging is the most direct cause of extrinsic aging and causes various skin damage phenomena including reduction of skin moisture due to skin barrier damage and the resultant skin dryness, reduced skin elasticity, skin roughness, pigmentation, increased epidermal thickness, etc.

The method generally used to alleviate the ultraviolet ray-induced photoaging, i.e., ultraviolet ray-induced skin barrier damage, is to supplement moisture and oils by applying a cosmetic including a sunscreen, a moisturizer, etc. or a preparation for external application to skin such as an ointment. However, there is a limitation in that the skin-moisturizing effect is only temporary because the preparation for external application to skin is not absorbed to the dermal layer.

Therefore, researches on inner beauty, i.e., beauty food, are increasing in order to overcome the limitation of the products for external application to skin and achieve the effect of alleviating systemic skin barrier damage. Korean Patent Publication No. 2006-0119384 discloses an oral composition for improving skin beauty, which contains a soybean extract powder and a red ginseng concentrate powder, Korean Patent Publication No. 2009-0054723 discloses an oral skin beauty composition containing curcumin as an active ingredient, and Korean Patent Publication No. 2016-0035219 discloses a health functional food for moisturizing skin, which contains tyndallized dead lactobacillus cells as an active ingredient.

Gaedarae (Actinidia polygama) is native to Korea, northeastern China, northeastern Russia and Japan. More than 30 similar species are reported in the genus Actinidia, representatively darae (Actinidia arguta), jwidarae (Actinidia kolomikta), seomdarae (Actinidia rufa), etc. Chamdarae (Actinidia chinensis), commonly known as kiwi, also belongs to the genus Actinidia. However, not all the plants in the genus Actinidia show the same or similar physiological activity or functionality.

Korean Patent Publication No. 2017-0056979 discloses a composition for whitening skin and a composition for improving wrinkles, which contain a gaedarae (Actinidia polygama) extract and a jwidarae (Actinidia kolomikta) as active ingredients, while presenting their effects of inhibiting the activity of tyrosinase and elastase in vitro. However, it does not describe the alleviation of ultraviolet ray-induced skin barrier damage.

And, Korean Patent Publication No. 2018-0041282 discloses a cosmetic composition for preventing or alleviating skin aging, which contains a mixture of the extracts of darae (Actinidia arguta), black berry and apricot kernel, while presenting its effect of inhibiting the expression of matrix metalloproteinases (MMPs) in vitro. However, there is difference in plant species from gaedarae (Actinidia polygama). It is also described that the inhibitory effect on the expression of MMPs is insignificant with darae (Actinidia arguta) alone and the alleviation of ultraviolet ray-induced skin barrier damage is not described.

### REFERENCES OF RELATED ART

### Patent Documents

Korean Patent Publication No. 2006-0119384.
Korean Patent Publication No. 2009-0054723.
Korean Patent Publication No. 2016-0035219.
Korean Patent Publication No. 2017-0056979.
Korean Patent Publication No. 2018-0041282.

### Non-Patent Documents

Sand, M., et al., Journal of Dermatological Science, 2009. 53(3): p. 169-175.
El-Domyati, M., et al., Experimental Dermatology, 2002. 11(5): p. 398-405
Lee, J. Y., et al., Journal of Dermatological Science, 2008. 50(2): p. 99-107

### DISCLOSURE

### Technical Problem

The present disclosure is directed to providing a food composition for alleviating ultraviolet ray-induced skin damage or moisturizing skin, which contains a gaedarae (Actinidia polygama) extract as an active ingredient.

The present disclosure is also directed to providing an animal feed composition for alleviating ultraviolet ray-induced skin damage or moisturizing skin, which contains a gaedarae (Actinidia polygama) extract as an active ingredient.

The present disclosure is also directed to providing a pharmaceutical composition for treating or preventing ultraviolet ray-induced skin damage, which contains a gaedarae (Actinidia polygama) extract as an active ingredient.

The present disclosure is also directed to providing a pharmaceutical composition for animals for treating or preventing ultraviolet ray-induced skin damage, which contains a gaedarae (Actinidia polygama) extract as an active ingredient.

The present disclosure is also directed to providing a cosmetic composition for alleviating ultraviolet ray-induced skin damage or moisturizing skin, which contains a gaedarae (Actinidia polygama) extract as an active ingredient.

The present disclosure is also directed to providing a method for treating ultraviolet ray-induced skin damage by administering the composition to human or a non-human animal.

The present disclosure is also directed to providing a novel use of a gaedarae (Actinidia polygama) extract for preparing a medication or a medication for animals for treating ultraviolet ray-induced skin damage.

### Technical Solution

The present disclosure provides a food composition for alleviating ultraviolet ray-induced skin damage or moisturizing skin, which contains a gaedarae (Actinidia polygama) extract as an active ingredient.

In an exemplary embodiment of the present disclosure, the ultraviolet ray-induced skin damage may be skin dryness, reduced skin elasticity or skin roughness.

In an exemplary embodiment of the present disclosure, the gaedarae (Actinidia polygama) extract may be an extract of gaedarae fruit.

In an exemplary embodiment of the present disclosure, the gaedarae (Actinidia polygama) extract may be an extract obtained with water, a C1-4 alcohol or a mixture solvent thereof.

In an exemplary embodiment of the present disclosure, the food composition may be formulated into a powder, a granule, a tablet, a capsule, a pill, an extract, a jelly, a tea bag or a beverage.

In an exemplary embodiment of the present disclosure, the food composition may be a health functional food for alleviating ultraviolet ray-induced skin damage.

In an exemplary embodiment of the present disclosure, the food composition may be a health functional food for moisturizing skin.

The present disclosure also provides an animal feed composition for alleviating ultraviolet ray-induced skin damage or moisturizing skin, which contains a gaedarae (Actinidia polygama) extract as an active ingredient.

The present disclosure also provides a pharmaceutical composition for treating or preventing ultraviolet ray-induced skin damage, which contains a gaedarae (Actinidia polygama) extract as an active ingredient.

The present disclosure also provides a pharmaceutical composition for animals for treating or preventing ultraviolet ray-induced skin damage, which contains a gaedarae (Actinidia polygama) extract as an active ingredient.

The present disclosure also provides a cosmetic composition for alleviating ultraviolet ray-induced skin damage or moisturizing skin, which contains a gaedarae (Actinidia polygama) extract as an active ingredient.

The present disclosure also provides a method for treating ultraviolet ray-induced skin damage by administering the composition to human or a non-human animal.

The present disclosure also provides a novel use of a gaedarae (Actinidia polygama) extract for preparing a medication or a medication for animals for treating ultraviolet ray-induced skin damage.

### Advantageous Effects

Since a composition containing a gaedarae (Actinidia polygama) extract as an active ingredient of the present disclosure can alleviate the reduction of skin moisture caused by ultraviolet ray-induced skin barrier damage and the resultant skin dryness, reduced skin elasticity and increased skin roughness, it can be utilized as a food composition for alleviating ultraviolet ray-induced skin damage or moisturizing skin and, furthermore, as a health functional food, an animal feed composition for alleviating skin damage or moisturizing skin, a pharmaceutical composition for treating skin damage, a pharmaceutical composition for animals for treating skin damage, or a cosmetic composition for alleviating skin damage or moisturizing skin.

### BRIEF DESCRIPTION OF DRAWINGS

FIG. 1 shows a result of comparing the DPPH radical-scavenging ability of Example 1, Comparative Example 1 and Comparative Example 2 of different darae species at different concentrations in Test Example 1.

FIG. 2 shows a result of comparing the ABTS radical-scavenging ability of Example 1, Comparative Example 1 and Comparative Example 2 of different darae species at different concentrations in Test Example 1.

FIG. 3 shows a result of, after treating human keratinocyte HaCaT cells with ultraviolet ray, treating with Example 1, Comparative Example 1 and Comparative Example 2 of different darae species and comparing MMP1 gene expression level with a normal control group and an induced group in Test Example 1.

FIG. 4 shows a result of, after treating human keratinocyte HaCaT cells with ultraviolet ray, treating with Example 1, Comparative Example 1 and Comparative Example 2 of different darae species and comparing MMP3 gene expression level with a normal control group and an induced group in Test Example 1.

FIG. 5 shows a result of, after treating human keratinocyte HaCaT cells with ultraviolet ray, treating with Example 1, Comparative Example 1 and Comparative Example 2 of different darae species and comparing collagen type I alpha 1 (COL1A1) gene expression level with a normal control group and an induced group in Test Example 1.

FIG. 6 shows a result of treating mouse-derived RAW264.7 macrophages in which inflammatory response is induced with LPS with Example 1, Comparative Example 1 and Comparative Example 2 of different darae species and comparing production of nitric oxide with a normal control group and an induced group in Test Example 1.

FIG. 7 shows a result of treating rat-derived RBL-2H3 mast cells with Example 1, Comparative Example 1 and Comparative Example 2 of different darae species and comparing production of interleukin 4 with a normal control group and an induced group in Test Example 1.

FIG. 8 shows a result of, after treating human keratinocyte HaCaT cells with ultraviolet ray, treating with Examples 1, 2 and 3 of different parts of gaedarae at 50 μg/mL and comparing cell death-inhibiting effect with a normal control group and an induced group in Test Example 2.

FIG. 9 shows a result of, after treating human keratinocyte HaCaT cells with ultraviolet ray, treating with Examples 1, 2 and 3 of different parts of gaedarae at 100 μg/mL and comparing cell death-inhibiting effect with a normal control group and an induced group in Test Example 2.

FIG. 10 shows a result of, after treating human keratinocyte HaCaT cells with ultraviolet ray, treating with Examples 1, 2 and 3 of different parts of gaedarae and comparing MMP1 gene expression level with a normal control group and an induced group in Test Example 2.

FIG. 11 shows a result of treating rat-derived RBL-2H3 mast cells treating with Examples 1, 2 and 3 of different parts of gaedarae and comparing production of interleukin 4 with a normal control group and an induced group in Test Example 2.

FIG. 12 shows a result of inducing skin damage by treating with ultraviolet ray and obtaining the images of skin 4 weeks later using a folliscope for a normal control group (Normal), an induced group (Saline), Example 1 (APWE) and Comparative Example 3 (HU-018) in Test Example 3.

FIG. 13 shows a result of inducing skin damage by treating with ultraviolet ray and obtaining the images of skin 6 weeks later using a Folliscope for a normal control group (Normal), an induced group (Saline), Example 1 (APWE) and Comparative Example 3 (HU-018) in Test Example 3.

FIG. 14 shows a result of inducing skin damage by treating with ultraviolet ray and obtaining 3D images and skin roughness images 4 weeks later using Primos Lite for a normal control group (Normal), an induced group (Saline), Example 1 (APWE) and Comparative Example 3 (HU-018) in Test Example 3.

FIG. 15 shows a result of inducing skin damage by treating with ultraviolet ray and obtaining 3D images and skin roughness images 6 weeks later using Primos Lite for a normal control group (Normal), an induced group (Saline), Example 1 (APWE) and Comparative Example 3 (HU-018) in Test Example 3.

### BEST MODE

Hereinafter, the present disclosure is described in detail.

The inventors of the present disclosure have identified that a gaedarae (Actinidia polygama) extract exhibits better effect of alleviating ultraviolet ray-induced skin damage or moisturizing skin than a darae (Actinidia arguta) extract and a chamdarae (Actinidia chinensis) extract. Specifically, they have identified that a gaedarae (Actinidia polygama) extract exhibits remarkably superior DPPH and ABTS radical-scavenging ability, exhibits superior effect of inhibiting ultraviolet ray-induced cell death in human keratinocyte HaCaT cells, remarkably reduces MMP1 and MMP3 gene expression in HaCaT cells induced by ultraviolet ray, remarkably increases COL1A1 gene expression, exhibits remarkably superior effect of inhibiting production of nitric oxide in mouse-derived RAW264.7 macrophages induced by LPS treatment and exhibits remarkably superior effect of inhibiting secretion of interleukin 4 in rat-derived RBL-2H3 mast cells, as compared to a darae (Actinidia arguta) extract and a chamdarae (Actinidia chinensis) extract.

In addition, the inventors of the present disclosure have identified, as a result of orally administering a gaedarae (Actinidia polygama) extract to a hairless mouse animal model in which skin barrier damage has been induced by ultraviolet ray and assessing skin moisture content, water loss, skin roughness, skin thickness, skin elasticity, etc., that the oral administration of a gaedarae extract provides an effect of alleviating ultraviolet ray-induced skin damage or moisturizing skin.

In addition, they have identified that the oral administration of a gaedarae (Actinidia polygama) extract is advantageous in terms of skin moisture content, water loss, skin elasticity, etc. as compared to transdermal administration.

The present disclosure relates to a composition for alleviating ultraviolet ray-induced skin damage or moisturizing skin, which contains a gaedarae (Actinidia polygama) extract as an active ingredient.

The gaedarae (Actinidia polygama) extract exhibits remarkably excellent effect of alleviating ultraviolet ray-induced skin damage or moisturizing skin as compared to the extracts of other plants in the genus Actinidia such as a darae (Actinidia arguta) extract, a jwidarae (Actinidia kolomikta) extract, a chamdarae (Actinidia chinensis) extract, etc.

The gaedarae (Actinidia polygama) extract may be an extract of the leaf, stem, fruit or whole plant of gaedarae (Actinidia polygama). However, a gaedarae fruit extract exhibits remarkably excellent effect of alleviating ultraviolet ray-induced skin damage or moisturizing skin. Specifically, although superior effects of inhibiting ultraviolet ray-induced cell death in human keratinocyte HaCaT cells, remarkably reducing MMP1 gene expression in HaCaT cells induced by ultraviolet ray and inhibiting secretion of interleukin 4 in rat-derived RBL-2H3 mast cells were observed for 250 μg/mL leaf, stem and fruit extracts of gaedarae (Actinidia polygama), the gaedarae (Actinidia polygama) fruit extract exhibited the most superior effect.

In addition, the gaedarae (Actinidia polygama) extract may be an extract obtained with water, a C1-4 alcohol or a mixture solvent thereof.

The water is not particularly limited as long as it is suitable for preparation of food. For example, underground water, purified water, distilled water, deionized water, etc. may be used.

The C1-4 alcohol is not particularly limited. For example, methanol, ethanol, propanol, butanol, n-propanol, isopropanol, n-butanol, etc., specifically ethanol, may be used.

The mixture solvent is not particularly limited. For example, as a mixture solvent of water and ethanol, a 5-95 wt % ethanol aqueous solution, a 10-90 wt % ethanol aqueous solution, a 20-80 wt % ethanol aqueous solution or a 30-70 wt % ethanol aqueous solution may be used.

The water extract may be prepared by extracting gaedarae with water at 10-100° C. for 2-60 hours, although not being necessarily limited thereto.

The alcohol extract or the extract of a mixture solvent of water and an alcohol may be prepared by extracting gaedarae with a 30-70 wt % ethanol aqueous solution at 20-70° C. for 2-48 hours, although not being necessarily limited thereto.

The extract of gaedarae (Actinidia polygama) obtained with water, a C1-4 alcohol or a mixture solvent thereof may include a fraction obtained by refractionating the extract obtained with water, a C1-4 alcohol or a mixture solvent thereof with an organic solvent. The organic solvent may be one or more organic solvent selected from a C1-4 alcohol, hexane, acetone, ethyl acetate, chloroform, diethyl ether, etc. Specifically, it may be hexane or ethyl acetate.

The term ‘extract’ used in the present disclosure includes an extract obtained by extracting the ingredients contained in gaedarae (Actinidia polygama) using the solvent described above, a fraction fractionated therefrom, a concentrate obtained by additionally concentrating the extract or fraction, a purified product obtained by purifying or separating the same, a dried product obtained by drying the extract, fraction, concentrate or purified product, or a powder obtained by pulverizing the same.

The purified product may be prepared by various additional purification methods such as passing through an ultrafiltration membrane having a molecular weight cut-off value, separation by various chromatography techniques (for separation based on size, charge, hydrophobicity or affinity).

The composition of the present disclosure, which contains a gaedarae (Actinidia polygama) extract as an active ingredient, may alleviate one or more skin damages caused by ultraviolet ray-induced skin barrier damage such as increased skin moisture loss, decreased skin moisture content, increased skin roughness, reduced skin elasticity, etc. And, the food composition of the present disclosure, which contains a gaedarae (Actinidia polygama) extract as an active ingredient, may improve skin moisturization by alleviating one or more of increased skin moisture loss and decreased skin moisture content.

The increased skin moisture loss means a state where the transepidermal water loss (g/m²h) measured with Tewameter has been increased by 10%, 20%, 30%, 40%, 50% or 60% or more as compared to a normal control group, and the alleviation of the increased skin moisture loss means that the skin moisture loss, which has been increased by ultraviolet ray, is decreased by 10%, 20%, 30%, 40%, 50% or 60% or more as compared to an induced group or to 90-120%, specifically 95-110%, of the skin moisture loss of a normal control group.

The decreased skin moisture content means a state where the skin moisture content (A.U.) measured with Corneometer has been decreased by 10%, 20%, 30%, 40%, 50% or 60% or more as compared to a normal control group, and the alleviation of the decreased skin moisture content means that the skin moisture content, which has been decreased by ultraviolet ray, is increased by 10%, 15%, 20%, 25%, 30% or 35% or more as compared to an induced group or to 80-110%, specifically 90-105%, of the skin moisture content of a normal control group.

The increased skin roughness means a state where, as a result of measuring one or more of Rₐ (average skin roughness), Rₘₐₓ (maximum skin roughness: the largest difference in skin height of evenly divided 5 zones) and Rₜ (maximum skin roughness: the difference of the highest and lowest skin surface) using Primos Lite which quantitatively measures skin roughness based on skin microstructure and skin height by refracting a parallel fringe with a slight difference in height on skin surface, any of Rₐ, Rₘₐₓ and Rₜ has been increased by 5%, 10%, 15%, 20%, 25% or 30% or more as compared to a normal control group, and the alleviation of the skin roughness means that the skin roughness, which has been increased by ultraviolet ray, is decreased by 5%, 10%, 15%, 20%, 25% or 30% or more as compared to an induced group or to 80-110%, specifically 90-105%, of the skin roughness of a normal control group.

The reduced skin elasticity means a state where the skin firmness (R7) measured with Cutometer has been decreased by 10%, 15%, 20%, 25%, 30% or 35% or more as compared to a normal control group, and the alleviation of the skin elasticity means that the skin elasticity, which has been decreased by ultraviolet ray, is increased by 10%, 15%, 20%, 25%, 30% or 35% or more as compared to an induced group or to 80-110%, specifically 90-105%, of the skin elasticity of a normal control group. Alternatively, the reduced skin elasticity means a state where the alpha value measured with Ballistometer has been increased by 10%, 20%, 30%, 40%, 50% or 60% or more as compared to a normal control group, and the alleviation of the skin elasticity means that the skin elasticity, which has been decreased by ultraviolet ray, is increased by 10%, 15%, 20%, 25%, 30% or 35% or more as compared to an induced group or to 100-200%, specifically 150-200%, of the elasticity of a normal control group.

The present disclosure relates to a food composition for alleviating ultraviolet ray-induced skin damage or moisturizing skin, which contains a gaedarae (Actinidia polygama) extract as an active ingredient.

The ‘food composition’ contains, in addition to the gaedarae (Actinidia polygama) extract as an active ingredient, food ingredients described in commonly used standards and regulations for preparation of food (‘food codes’) and food additives described in food additive codes.

The additional ingredient includes, for example, a protein, a carbohydrate, a fat, a nutrient, a condiment and a flavorant, although not being specially limited thereto. As the carbohydrate, a monosaccharide, e.g., glucose, fructose, etc., a disaccharide, e.g., maltose, sucrose, lactose, etc., an oligosaccharide or a polysaccharide, e.g., dextrin, starch syrup, cyclodextrin, etc., a sugar alcohol, e.g., xylitol, sorbitol, erythritol, etc. may be used. As the flavorant, a natural flavorant (thaumatin or stevia extract (e.g., rubusoside A, glycyrrhizin, etc.)) or a synthetic flavorant (saccharin, aspartame, etc.) may be used.

When a food composition is prepared using the gaedarae (Actinidia polygama) extract as an active ingredient, the gaedarae (Actinidia polygama) extract may be contained with a content of, for example, 0.1-99 wt %, 0.5-95 wt %, 1-90 wt %, 2-80 wt %, 3-70 wt %, 4-60 wt % or 5-50 wt %, although the content is not limited specially as long as the effect of alleviating ultraviolet ray-induced skin damage or moisturizing skin is achieved.

The administration dosage of the gaedarae (Actinidia polygama) extract in the food composition as an active ingredient may be determined adequately by those of ordinary skill although it varies depending on the condition or body weight of a subject, the presence or absence of a disease and the period of administration. For example, 1-5,000 mg, specifically 5-2,000 mg, more specifically 10-1,000 mg, further more specifically 20-800 mg, most specifically 50-500 mg, may be administered per day. The number of administration is not limited specially and may be adjusted by those of ordinary skill within a range from three times a day to once a week. The administration dosage may be decreased in case of long-term intake for the purpose of health and hygiene improvement or health control.

The food composition may be formulated into, for example, a powder, a granule, a tablet, a capsule, a pill, an extract, a jelly, a tea bag or a beverage, although not being specially limited thereto.

In addition, the gaedarae (Actinidia polygama) extract may be added to a general food to provide the functionality of alleviating ultraviolet ray-induced skin damage or moisturizing skin. The food to which the extract may be added includes, for example, confectionery, bread or rice cakes, processed cocoa products or chocolates, processed meat or egg products, processed fish meat products, soybean curds or muk, noodles, teas, coffee, beverages, foods for special dietary uses, soy sauces or pastes, seasoned foods, dressings, kimchis, salted and fermented seafood products, pickled foods, boiled foods, alcoholic beverages, dried fish products, other foods, etc. exemplified in the Standards and Regulations of Foods (‘Food Code’) of Article 7 of the Food Sanitation Act, although not being specially limited thereto. In addition, it may be added to the processed milk products, processed meat products, packaged meats and processed egg products exemplified in the Processing Standards and Ingredient Specifications for Livestock Products of (‘Livestock Products Code’) Article 4 of the Livestock Products Sanitary Control Act.

The food composition containing the gaedarae (Actinidia polygama) extract as an active ingredient may be used alone as a “health functional food which helps to maintain the health of skin against ultraviolet ray-induced skin damage” or as a “health functional food which helps to moisturize skin”.

The ‘health functional food’ refers to a food prepared (or processed) according to legal standards using materials or ingredients having functions useful for the human body (Article 3(1) of the Health Functional Foods Act). The term ‘health functional food’ may correspond to the ‘dietary supplement’ of the US, the ‘food supplement’ of Europe, the ‘health functional food’ or ‘food for special health use (FoSHU)’ of Japan, the ‘health food’ of China, etc.

The food composition or the health functional food may further contain a food additive, and the appropriateness as the food additive is pursuant to the standards and criteria of the general rules, general test methods, etc. of the ‘Food Additive Code’.

The health functional food may contain, in addition to the gaedarae (Actinidia polygama) extract, a health functional food ingredient related with skin health such as fermented honeybush extract powder, pine bark extract, red ginseng/torilis fructus/corni fructus complex extract, fingerroot extract powder, probiotic HY7714, konjac potato extract (powder), rice bran extract, lithospermi radix extract powder, AP collagen peptide, dandelion complex extract, Collactive collagen peptide, low-molecular-weight collagen peptide, corn germ extract, fermented bean/barley mixture, wheat germ oil extract, pomegranate concentrate, N-acetylglucosamine, spirulina, chloroplast-containing plant, chlorella, phosphatidylserine, hyaluronic acid, aloe gel, etc. as a “health functional food which helps to maintain the health of skin against ultraviolet ray-induced skin damage” or as a “health functional food which helps to moisturize skin”.

The present disclosure relates to an animal feed composition for alleviating ultraviolet ray-induced skin damage or moisturizing skin, which contains a gaedarae (Actinidia polygama) extract as an active ingredient.

The ‘animal feed composition’ may contain, in addition to the gaedarae (Actinidia polygama) extract as an active ingredient, food ingredients described in the Standards and Regulations of Foods (‘Food Code’) and food additives described in the Food Additive Code. In addition, the feed materials listed in Table 1 and the complementary feeds listed in Table 2 of the ‘Standards and Regulations of Feeds, etc.’ may be used.

The ‘animal feed composition’ may be an extract from among the complementary feeds according to the ‘Standards and Regulations of Feeds, etc.’, and may be a complete feed including the complementary feed.

When an animal feed composition is prepared using the gaedarae (Actinidia polygama) extract as an active ingredient, the content of the gaedarae (Actinidia polygama) extract may be, for example, 0.1-99 wt %, 0.5-95 wt %, 1-90 wt %, 2-80 wt %, 3-70 wt %, 4-60 wt % or 5-50 wt % although it is not specially limited as long as the effect of alleviating ultraviolet ray-induced skin damage or moisturizing skin can be achieved. The administration dosage of the gaedarae (Actinidia polygama) extract in the animal feed composition as an active ingredient may be determined adequately by those of ordinary skill although it varies depending on the condition or body weight of a subject animal, the presence or absence of a disease and the period of administration. For example, 1-5,000 mg, specifically 5-2,000 mg, more specifically 10-1,000 mg, further more specifically 20-800 mg, most specifically 50-500 mg, may be administered per day. The number of administration is not limited specially and may be adjusted by those of ordinary skill within a range from three times a day to once a week. The administration dosage may be decreased in case of long-term intake for the purpose of health and hygiene improvement or health control.

The present disclosure relates to a pharmaceutical composition for treating or preventing ultraviolet ray-induced skin damage, which contains a gaedarae (Actinidia polygama) extract as an active ingredient.

The present disclosure also relates to a pharmaceutical composition for animals for treating or preventing ultraviolet ray-induced skin damage, which contains a gaedarae (Actinidia polygama) extract as an active ingredient.

The present disclosure provides a method for treating ultraviolet ray-induced skin damage by administering the composition to human or a non-human animal.

The present disclosure also provides a novel use of a gaedarae (Actinidia polygama) extract for preparing a medication or a medication for animals for treating ultraviolet ray-induced skin damage.

The ‘pharmaceutical composition’, ‘medication’, ‘pharmaceutical composition for animals’ or ‘medication for animals’ may further contain, in addition the gaedarae (Actinidia polygama) extract as an active ingredient, an adequate carrier, excipient or diluent commonly used for preparation of a pharmaceutical composition, etc.

The ‘carrier’ is a compound which allows easy delivery of a target compound into a cell or tissue. The ‘diluent’ is a compound which stabilizes a biologically active form of a target compound and is dissolved in water in which the compound is to be dissolved.

The carrier, excipient or diluent may be, for example, lactose, glucose, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinylpyrrolidone, water, methyl hydroxybenzoate, propyl hydroxybenzoate, talc, magnesium stearate, mineral oil, etc., although not being specially limited thereto.

The administration dosage of the pharmaceutical composition, medication, pharmaceutical composition for animals or medication for animals may vary depending on the age, sex and body weight of a patient or an animal to be treated, and may be dependent, above all, on the condition of the subject to be treated, the particular category or type of the disease to be treated, administration route, the characteristics of the therapeutic agent used.

The administration dosage of the pharmaceutical composition, medication, pharmaceutical composition for animals or medication for animals may be determined adequately depending on the absorption rate and excretion rate of the active ingredient in the body, the age, body weight, sex and condition of a patient or animal to be treated, the severity of a disease to be treated, etc. It may be administered with a daily dosage of generally 0.1-1,000 mg/kg, specifically 1-500 mg/kg, more specifically 5-250 mg/kg, most specifically 10-100 mg/kg. The formulated unit dosage form may be administered several times with regular intervals.

The pharmaceutical composition, medication, pharmaceutical composition for animals or medication for animals may be administered individually as a prophylactic or therapeutic agent or in combination with another therapeutic agent either sequentially or simultaneously.

The pharmaceutical composition, medication, pharmaceutical composition for animals or medication for animals may be formulated into an oral formulation such as a powder, a granule, a tablet, a capsule, a troche, a suspension, an emulsion, a syrup, an aerosol, etc., or a parenteral formulation such as a sterilized aqueous solution, a nonaqueous solution, a suspension, an emulsion, a freeze-dried formulation, a suppository, etc. according to common methods.

The formulation may be prepared using a commonly used diluent or excipient such as a filler, an extender, a binder, a wetting agent, a disintegrant, a surfactant, etc.

Solid formulations for oral administration include a tablet, a pill, a powder, a granule, a capsule, a troche, etc. and may be prepared by mixing the gaedarae (Actinidia polygama) extract with at least one excipient, e.g., starch, calcium carbonate, sucrose, lactose, gelatin, etc. In addition to the simple excipient, a lubricant such as magnesium stearate and talc may also be used. Liquid formulations for oral administration include a suspension, an internal solution, an emulsion, a syrup, etc., and may contain various excipients, e.g., a wetting agent, a sweetener, an aromatic, a preservative, etc. in addition to a commonly used simple diluent such as water or liquid paraffin.

Formulations for parenteral administration may use, as a nonaqueous solvent or a suspension medium, propylene glycol, polyethylene glycol, vegetable oil such as olive oil, an injectable ester such as ethyl oleate, etc. As a base of a suppository, witepsol, macrogol, Tween 61, cocoa butter, laurin butter, glycerogelatin, etc. may be used.

The present disclosure relates to a cosmetic composition for alleviating ultraviolet ray-induced skin damage or moisturizing skin, which contains a gaedarae (Actinidia polygama) extract as an active ingredient.

The cosmetic composition may be formulated into a cream such as a nourishing cream, an eye cream, a massage cream or a cleansing cream, a pack, a lotion such as a nourishing lotion, an essence, a toilet water such as a softening toilet water or nourishing toilet water, a powder, a foundation, a makeup base, etc. However, the present disclosure is not limited to the formulations exemplified above as long as the purpose of the present disclosure can be achieved. In addition, the cosmetic composition according to the present disclosure may be formulated according to a common preparation method.

The cosmetic composition of the present disclosure may be formulated into any formulation selected from a group consisting of a skin lotion, a skin softener, a skin toner, an astringent, a lotion, a milk lotion, a moisturizing lotion, a nourishing lotion, a massage cream, a nourishing cream, a moisturizing cream, a hand cream, an essence, a pack, a mask pack, a mask sheet, an exfoliant, a soap, a shampoo, a cleansing foam, a cleansing lotion, a cleansing cream, a body lotion, a body cleanser, an emulsion, a pressed powder, a loose powder and an eye shadow, although not being specially limited thereto.

The effective content of the gaedarae (Actinidia polygama) extract in the cosmetic composition may 0.0001-20 wt % based on the total weight of the composition, although not being specially limited thereto. In addition to the gaedarae (Actinidia polygama) extract, the cosmetic composition may further contain other additives such as an excipient, a carrier, etc. and common ingredients included in general skin cosmetics as desired.

The cosmetic composition may further contain a transdermal penetration enhancer. The term transdermal penetration enhancer used in the present disclosure refers to an agent which allows a desired ingredient to penetrate into the vascular cells of skin with high absorption rate. Specifically, it includes other phospholipid ingredients, liposome ingredients, etc. used in lecithin cosmetics, although not being limited thereto.

In addition, one or more oil selected from a vegetable oil, a mineral oil, a silicone oil and a synthetic oil may be used for an oil phase. More specifically, mineral oil, cyclomethicone, squalane, octyldodecyl myristate, olive oil, Vitis vinifera seed oil, macadamia nut oil, glyceryl octanoate, castor oil, ethylhexyl isononanoate, dimethicone, cyclopentasiloxane, sunflower seed oil, etc. may be used.

In addition, 0.1-5 wt % of a surfactant, a higher alcohol, etc. may be used to enhance emulsification ability. As the surfactant, common surfactants such as a non-ionic surfactant, an anionic surfactant, a cationic surfactant, an amphoteric surfactant, a phospholipid, etc. may be used. Specifically, sorbitan sesquioleate, polysorbate 60, glyceryl stearate, lipophilic glyceryl stearate, sorbitan oleate, sorbitan stearate, DEA-cetyl phosphate, sorbitan stearate/cetyl phosphate, glyceryl stearate/polyethylene glycol 100 stearate, ceteareth-6 olivate, arachidyl alcohol/behenyl alcohol/arachidyl glucoside, polypropylene glycol-26-buteth-26/polyethylene glycol-40 hydrogenated castor oil, etc. may be used. As the higher alcohol, a C12-20 alcohol, e.g., cetyl alcohol, stearyl alcohol, octyldodecanol, isostearyl alcohol, etc. may be used either alone or in combination.

As an aqueous phase ingredient for regulation of the viscosity or hardness of an aqueous phase, 0.001-5 wt % of one or more thickener such as carbomer, xanthan gum, bentonite, magnesium aluminum silicate, cellulose gum, dextrin palmitate, etc. may be further added.

In addition, a sunscreen, an antioxidant (butylhydroxyanisole, propyl gallate, erythorbic acid, tocopheryl acetate, butylated hydroxytoluene, etc.), an antiseptic (methylparaben, butylparaben, propylparaben, phenoxyethanol, imidazolidinyl urea, chlorphenesin, etc.), a colorant, a pH adjuster (triethanolamine, citric acid, sodium citrate, malic acid, sodium malate, fumaric acid, sodium fumarate, succinic acid, sodium succinate, sodium hydroxide, dibasic sodium phosphate, etc.), a moisturizer (glycerin, sorbitol, propylene glycol, butylene glycol, hexylene glycol, diglycerin, betaine, glycereth-26, methyl gluceth-20, etc.), a lubricant, etc. may further added to the cosmetic composition of the present disclosure together with a medically effective ingredient such as a higher fatty acid, a vitamin, etc., as desired.

In addition, the cosmetic composition of the present disclosure may further contain a substance that can supplementarily provide essential nutrients to skin.

Specifically, it may contain an adjuvant such as a natural flavor, a cosmetic flavor or a medicinal herb, although not being limited thereto.

The method for treating ultraviolet ray-induced skin damage includes administering the composition to a human or a non-human animal, particularly a mammal. Specifically, the composition may be orally administered to a subject having ultraviolet ray-induced skin damage.

The subject having ultraviolet ray-induced skin damage may be a subject having increased skin moisture loss, decreased skin moisture content, increased skin roughness or reduced skin elasticity.

For the administration dosage, administration method and number of administration for the treatment, the foregoing description about the administration dosage, administration method and number of administration for the pharmaceutical composition, medication, pharmaceutical composition for animals or medication for animals may be consulted.

Hereinafter, the present disclosure is described in more detail through specific examples. However, the following examples are only for illustrating the present disclosure more specifically, and it will be obvious to those having ordinary knowledge in the art that the scope of the present disclosure is not limited by them.

### Preparation Example: Preparation of Extracts

After extracting 3.2 kg of each of dried gaedarae fruit, dried darae fruit, dried chamdarae fruit, dried gaedarae leaf and dried gaedarae stem at 85±5° C. for 3-5 hours by adding 12-14 equivalents of purified water based on weight, the extract was filtered through a 1-μm filter and then concentrated to a solid content of 40-50 wt % at 65° C. or below using a vacuum evaporator. The concentrated extract was sterilized at 85±5° C. for 30-60 minutes, packaged into a plastic bottle and then stored in a refrigerator for use in experiments.

The prepared gaedarae (Actinidia polygama) fruit extract (Example 1, APWE) was a brown soft extract and had a solid content of 45.9 wt %. And, the prepared darae (Actinidia arguta) fruit extract (Comparative Example 1), chamdarae (Actinidia chinensis) fruit extract (Comparative Example 2), gaedarae (Actinidia polygama) leaf extract (Example 2) and gaedarae (Actinidia polygama) stem extract (Example 3) had solid contents of 42.5 wt %, 17 wt %, 4.15 wt % and 1.62 wt %, respectively.

### Test Example 1: Comparison of Plants in the Genus Actinidia

For comparison of the effect of alleviating ultraviolet ray-induced skin damage or moisturizing skin of the gaedarae (Actinidia polygama) fruit extract of Preparation Example (Example 1, APWE) with darae (Actinidia arguta) fruit extract (Comparative Example 1) and chamdarae (Actinidia chinensis) fruit extract (Comparative Example 2) of the same genus, DPPH and ABTS radical-scavenging ability, the effect of decreased MMP1 and MMP3 gene expression and increased COL1A1 gene expression in human keratinocyte HaCaT cells induced by ultraviolet ray, the effect of inhibited production of nitric oxide in mouse-derived RAW264.7 macrophages caused by treatment with LPS and the effect of inhibited secretion of interleukin 4 in rat-derived RBL-2H3 mast cells were investigated.

1. Measurement of DPPH and ABTS Radical-Scavenging Ability

DPPH [2,2-Di(4-tert-octylphenyl)-1-picrylhydrazyl] is a free-radical compound. It is a violet compound which is dissolved in an organic solvent and exhibits maximum absorption at 520-540 nm. The DPPH compound exhibits as a violet DPPH radical (DPPH) when dissolved and becomes colorless when reduced to DPPH by accepting an electron from an antioxidant. Accordingly, antioxidant activity can be compared from the ratio of decolorized DPPH radicals. After mixing 50 μL of a 0.36 mM DPPH solution and 50 μL of a test substance dissolved in ethanol at 1:1 and conducting reaction at room temperature in the dark for 30 minutes, absorbance was measured at 540 nm.

The DPPH radical-scavenging ability of Example 1, Comparative Example 1 and Comparative Example 2 of different darae species at different concentrations was calculated from the measured absorbance. The result is shown in FIG. 1. Half-maximal inhibitory concentration (IC₅₀) calculated based on this result is shown in Table 1.

ABTS [2,2-azino-bis(3-ethylbenzthiazoline-6-sulfonic acid)] exhibits pale bluish green color when dissolved in distilled water. When mixed with potassium persulfate at 1:1, it is oxidized, thereby exhibiting dark bluish green color and maximum absorbance at 732 nm. After dissolving 7.4 mM 2,2-azino-bis(3-ethylbenzthiazoline-6-sulfonic acid) and 2.6 mM potassium persulfate respectively in distilled water and mixing at 1:1, ABTS⁺ is formed by conducting reaction at room temperature in the dark for 24 hours. 24 hours later, the ABTS solution was diluted to purified water to adjust absorbance at 732 nm to 0.7. Then, after adding 10 μL of a test substance to 190 μL of the ABTS solution and conducting reaction in the dark for 10 minutes, absorbance was measured at 732 nm.

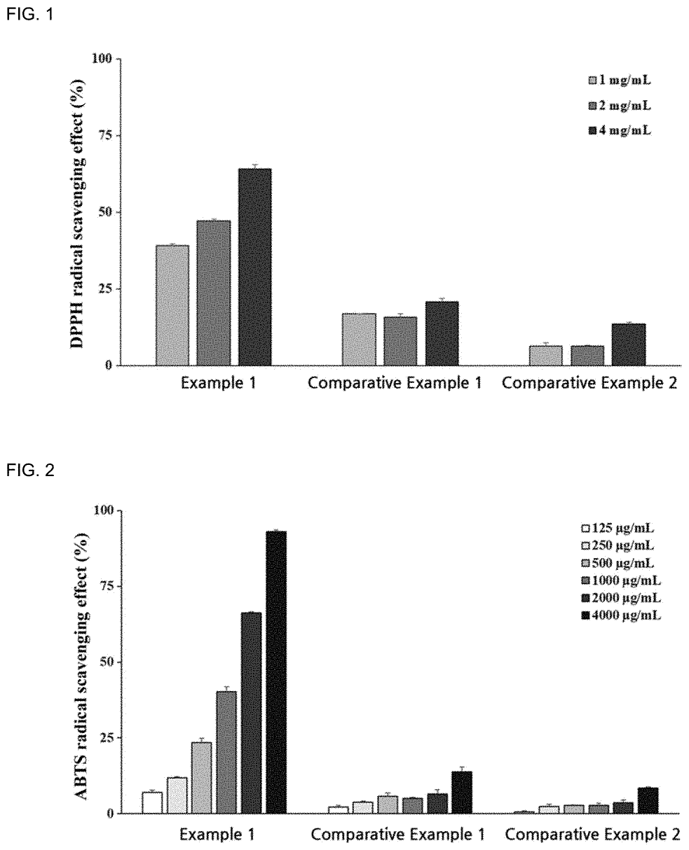

The ABTS radical-scavenging ability of Example 1, Comparative Example 1 and Comparative Example 2 of different darae species at different concentrations was calculated from the measured absorbance. The result is shown in FIG. 2. IC₅₀ is shown in Table 1.

TABLE 1

| | DPPH | | ABTS |
|---|---|---|---|
| | radical-scavenging | | radical-scavenging |
| | ability IC₅₀ | | ability IC₅₀ |
| L-Ascorbic | 0.03 ± 0.01 | Trolox | 0.10 ± 0.01 |
| acid | | | |
| Example 1 | 2.33 ± 0.06*** | Example 1 | 1.31 ± 0.03*** |
| Comparative | 16.41 ± 2.10** | Comparative | 13.40 ± 1.54** |
| Example 1 | | Example 1 | |
| Comparative | 11.98 ± 2.00** | Comparative | 21.20 ± 2.16** |
| Example 2 | | Example 2 | |

**p < 0.01;
***p < 0.001 vs L-ascorbic acid, Trolox (unit: mg/mL)

The DPPH radical-scavenging ability of the gaedarae fruit extract (Example 1) was increased with concentration. High activity of 39-64% was achieved at concentrations of 1-4 mg/mL. In contrast, the darae fruit extract (Comparative Example 1) and the chamdarae fruit extract (Comparative Example 2) did not show concentration-dependent increase in radical-scavenging ability at 1-2 mg/mL. They showed relatively low activity of 21% and 14%, respectively, at 4 mg/mL. In addition, Example 1 and Comparative Examples 1 and 2 showed IC₅₀, i.e., the concentration at which 50% of radical-scavenging ability is achieved, of 2.33, 16.41 and 11.98 mg/mL, respectively. That is to say, Example 1 showed the most superior radical-scavenging ability.

A similar result was observed for ABTS radical-scavenging ability as the DPPH radical-scavenging ability. Example 1 showed radical-scavenging ability of 7.0-93.0% at 125-4000 μg/mL, whereas Comparative Examples 1 and 2 showed low activity of 2.1-13.8% and 0.5-8.5%, respectively. When the ABTS radical-scavenging ability was investigated with IC₅₀, Example 1 showed the lowest value of 1.31 mg/mL, whereas Comparative Examples 1 and 2 showed very high values of 13.40 and 21.20 mg/mL, respectively. That is to say, Example 1 showed the most excellent ABTS radical-scavenging ability.

2. Measurement of Effect of Inhibiting Ultraviolet Ray-Induced Skin Damage in Human Keratinocyte HaCaT Cells

Human keratinocyte HaCaT cells, which are favorable in terms of the interpretation and assessment of the result of skin damage-related tests, were acquired from Sungkyun Biotech. The cells were cultured in an incubator set to 37° C. and 5% CO₂. The cells were cultured on a 75-cm² flask to 1×10⁷ cells/flask using Dulbecco's minimum essential medium (DMEM) supplemented with 10% fetal bovine serum (FBS), 1% L-glutamine, 1% HEPES, 100 units/mL penicillin and 100 μg/mL streptomycin. The medium was replaced with a fresh medium once in three days.

Ultraviolet ray (UVB) was irradiated to the HaCaT cells with an intensity of 30 mJ/cm². Damage of the HaCaT cells was induced by irradiating UVB using VL-215.LM (Vilber Lourmat, France) before treating with a sample. After the ultraviolet ray irradiation, the cells were treated with the gaedarae fruit extract (Example 1), the darae fruit extract (Comparative Example 1) or the chamdarae fruit extract (Comparative Example 2). A normal control group was not treated with UVB, and an induced group was irradiated with UVB.

For analysis of gene expression level, the HaCaT cells were cultured after seeding onto a 6-well cell culture plate at 1×10⁶ cells/well. After filling 1×PBS in each well, UVB was irradiated with an intensity of 30 mJ/cm². After the ultraviolet ray irradiation, 2 mL of the test substance diluted in a medium was added and the cells were cultured at 37° C. for 24 hours. 24 hours later, RNA was obtained and cDNA was synthesized using a LaboPass cDNA synthesis kit. The MMP1, MMP3 and COL1A1 gene expression level of the HaCaT cells was analyzed using the synthesized cDNA by real-time PCR.

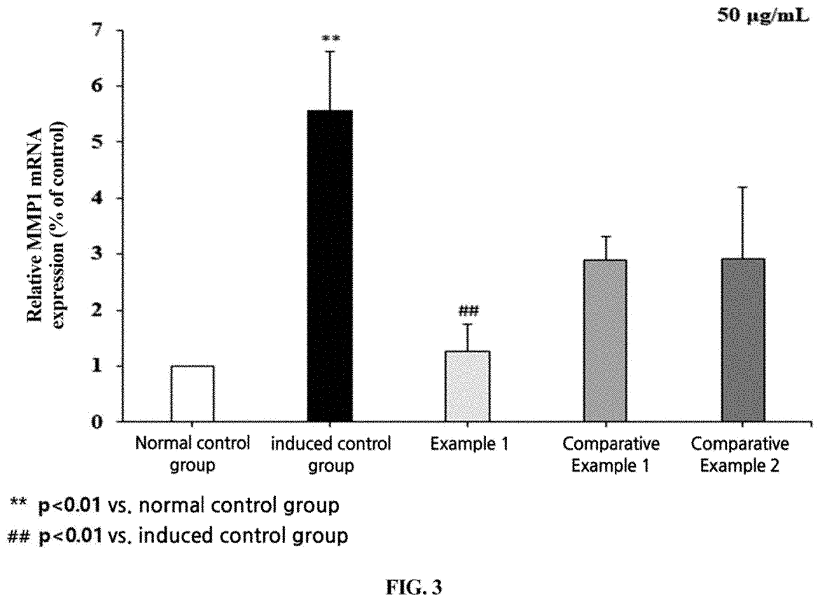

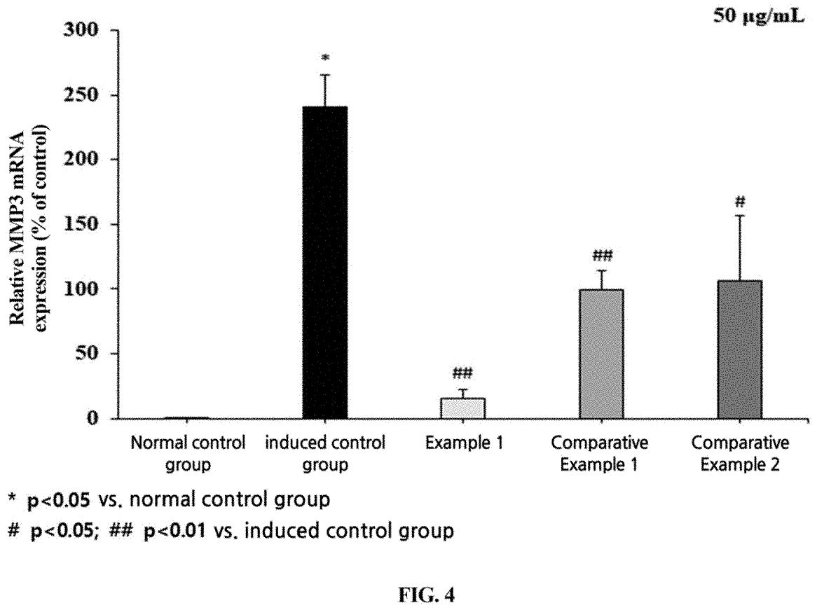

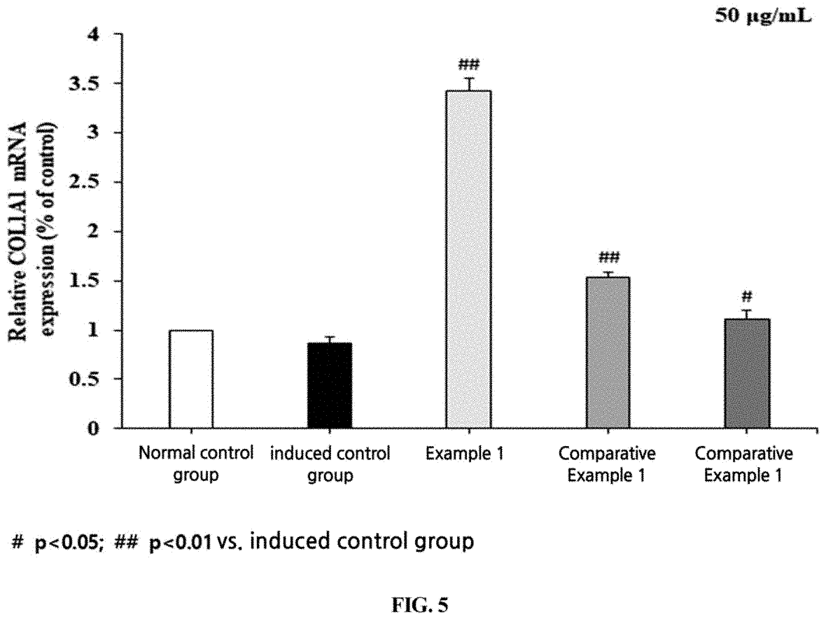

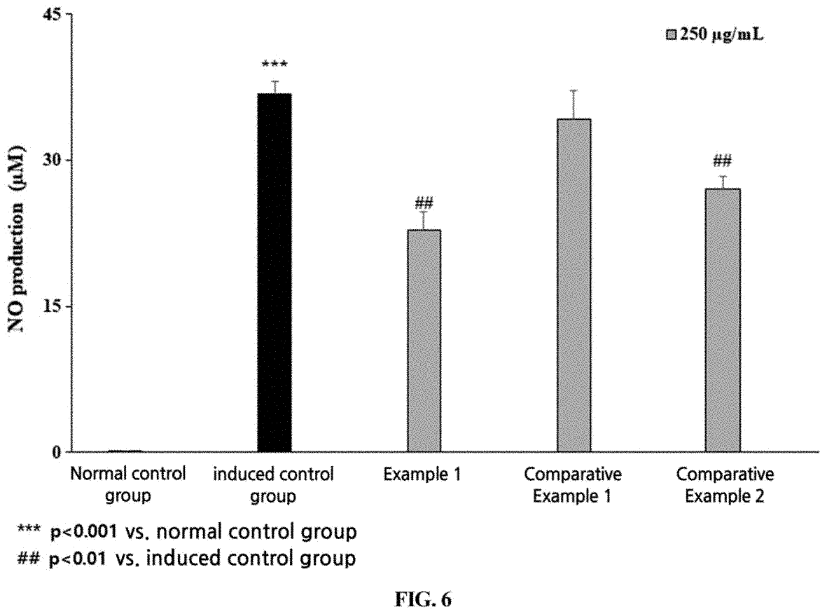

The result of, after treating the human keratinocyte HaCaT cells with ultraviolet ray and treating the cells with Example 1, Comparative Example 1 and Comparative Example 2 of different darae species, comparing the gene expression level with the normal control group and the induced group is shown in FIG. 3 (MMP1), FIG. 4 (MMP3) and FIG. 5 (COL1A1).

As shown in FIG. 3, among the extracts of different darae species, the gaedarae fruit extract (Example 1) showed the largest decrease in the MMP1 gene expression level by about 87% with respect to the induced group, followed by 47% decrease for the darae fruit extract (Comparative Example 1) and 37% decrease for the chamdarae fruit extract (Comparative Example 2).

As shown in FIG. 4, among the extracts of different darae species, the gaedarae fruit extract (Example 1) showed the largest decrease in the MMP3 gene expression level by about 93% with respect to the induced group, followed by 58% for the darae fruit extract (Comparative Example 1) and 45% decrease for the chamdarae fruit extract (Comparative Example 2).

As shown in FIG. 5, among the extracts of different darae species, the gaedarae fruit extract (Example 1) showed the largest increase in the COL1A1 gene expression level by 396% with respect to the induced group, followed by 177% increase for the darae fruit extract (Comparative Example 1) and 128% increase for the chamdarae fruit extract (Comparative Example 2).

As a result of treating human keratinocyte HaCaT cells with ultraviolet ray and analyzing the MMP1, MMP3 and COL1A1 gene expression level after treating with Example 1, Comparative Example 1 and Comparative Example 2 of different darae species, it was confirmed that the gaedarae fruit extract exhibits the most excellent skin-protecting effect among the different darae species.

3. Measurement of Effect of Inhibited Production of Nitric Oxide in Mouse-Derived RAW264.7 Macrophaqes Caused by Treatment with LPS

Mouse-derived RAW264.7 macrophages were purchased from American Type Culture Collection (ATCC, USA). The cells were cultured in RPMI-1640 medium (Sigma, USA) supplemented with 10% heat-inactivated fetal bovine serum (FBS), 1% L-glutamine, 1% HEPES, 100 units/mL penicillin and 100 μg/mL streptomycin under a humidified environment of 5% CO₂ and 37° C. After suspending the cells using a cell scraper to resolve overpopulation due to cell proliferation, the cells were subcultured and those at passages 6-9 were used for experiment.

For assessment of the antiinflammatory effect of the treatment with the extract using the mouse-derived RAW264.7 macrophages, the cells were seeded onto a 24-well plate at 3×10⁵ cells/well and cultured for 18 hours. After removing the culture medium, the cells were treated with the extract diluted in serum-free medium at a concentration of 250 μg/mL. At the same time, the cells were treated with LPS diluted in serum-free medium at a final concentration of 500 ng/mL for 24 hours. After the culturing, the culture was centrifuged (5,000 rpm, 3 minutes, 4° C.) to remove suspending cells and the amount of nitric oxide (NO), which is an inflammatory mediator, secreted by LPS was measured from the supernatant.

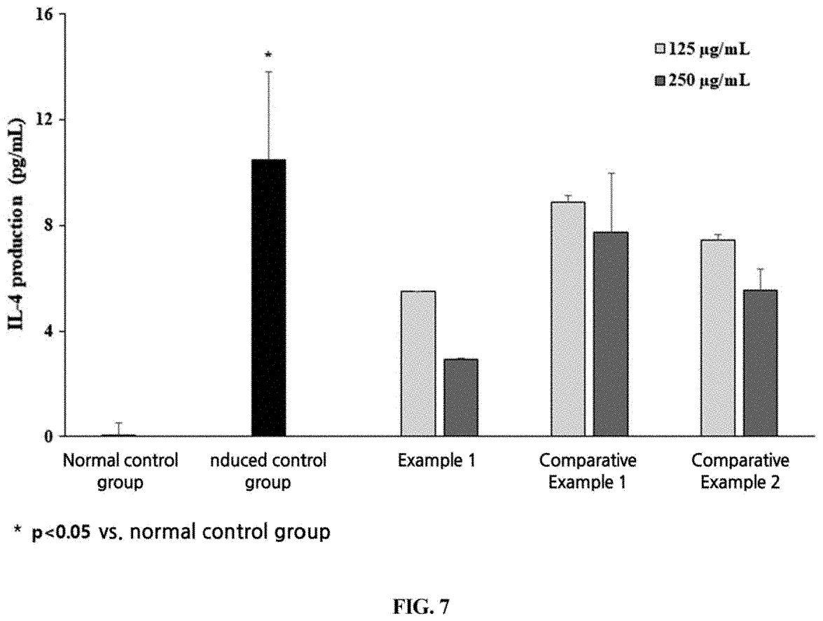

The secretion amount of nitric oxide was quantified by reacting a mixture of 50 μL of Griess's reagent and 50 μL of the supernatant on a 96-well plate for 15 minutes at room temperature and measuring absorbance at 540 nm using a microplate reader. The result is shown in FIG. 6.

When macrophages are treated with LPS, NF-κB is activated during signal transduction as Toll-like receptor is activated by bacterial stimulation. As a result, the production of inflammatory mediator nitric oxide from L-arginine is increased as the expression of inducible nitric oxide synthase (iNOS) is increased. The secretion amount of nitric oxide due to inflammatory response induced by treatment with 500 ng/mL LPS was measured to be 36 μM for the induced group. The amount was 38, 7 and 26%, respectively, when treated with Example 1, Comparative Examples 1 and 2 at a final concentration of 250 μg/mL. That is to say, Example 1 showed the highest effect of inhibiting the production of nitric oxide.

Accordingly, it was confirmed that the gaedarae fruit extract exhibits remarkably superior effect of inhibiting inflammation as compared to other darae species, with excellent effect of inhibiting the secretion of nitric oxide in RAW264.7 cells in which inflammatory response was induced with LPS.

4. Measurement of Effect of Inhibited Secretion of Interleukin 4 in Rat-Derived RBL-2H3 Mast Cells

Rat-derived RBL-2H3 mast cells were purchased from American Type Culture Collection (ATCC, USA). The cells were cultured in Eagle's minimum essential medium (EMEM, ATCC, USA) supplemented with 15% heat-inactivated FBS, 100 units/mL penicillin and 100 μg/mL streptomycin under a humidified environment of 5% CO₂ and 37° C. After suspending the cells by treating with a 0.05% trypsin-EDTA solution to resolve overpopulation due to cell proliferation, the cells were subcultured and those at passages 4-5 were used for experiment.

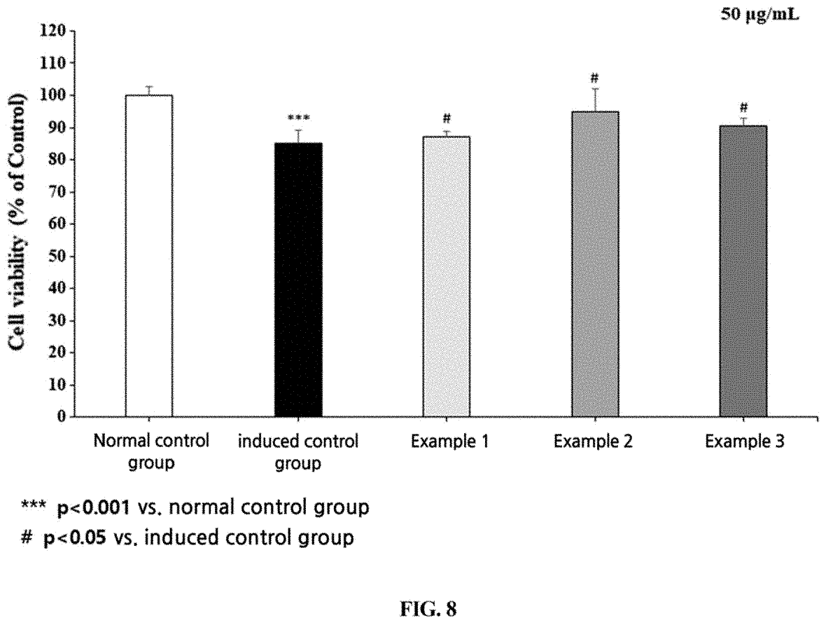

In order to measure cell viability and IL-4 production in the RBL-2H3 cells depending on treatment with a sample, the cells were seeded onto a 24-well plate at 2×10⁵ cells/well and cultured for 24 hours. After removing the culture medium, the cells were treated with a sample diluted in a serum-free medium at different concentrations. At the same time, the mast cells were sensitized by treating with 1 μM A23187 and 50 nM PMA for 18 hours. After the culturing, the culture was centrifuged (5,000 rpm, 3 minutes, 4° C.) and the secreted interleukin 4 (IL-4) was quantified using an ELISA kit (KOMA BIOTECH Co., Korea). The result is shown in FIG. 7.

The secretion amount of IL-4 of the induced group was 10.5 μg/mL, and the secretion amount was decreased as the sample concentration was increased. The effect of inhibiting IL-4 secretion of Example 1 and Comparative Examples 1 and 2 at 250 μg/mL was 72, 26 and 47%, respectively. That is to say, Example 1 showed the highest effect of inhibiting IL-4 secretion.

Accordingly, through comparison of the secretion amount of the allergic reaction-inducing cytokine IL-4 in rat-derived RBL-2H3 mast cells, it was confirmed that the gaedarae fruit extract exhibits remarkably superior effect of inhibiting IL-4 secretion as compared to other darae species.

### Test Example 2: Comparison of Different Parts of Gaedarae (Actinidia polygama)

For comparison of the effect of alleviating ultraviolet ray-induced skin damage or moisturizing skin of the gaedarae (Actinidia polygama) fruit extract of Preparation Example (Example 1, APWE), the gaedarae leaf extract (Example 2) and the gaedarae stem extract (Example 3), the effect of inhibiting ultraviolet ray-induced cell death in human keratinocyte HaCaT cells, the effect of reducing MMP1 gene expression and the effect of inhibiting interleukin 4 secretion in rat-derived RBL-2H3 mast cells were investigated.

1. Measurement of Effect of Inhibiting Ultraviolet Ray-Induced Skin Damage in Human Keratinocyte HaCaT Cells

Preparation of human keratinocyte HaCaT cells, irradiation with UVB and analysis of MMP1 gene expression level were conducted in the same manner as in 2 of Test Example 1.

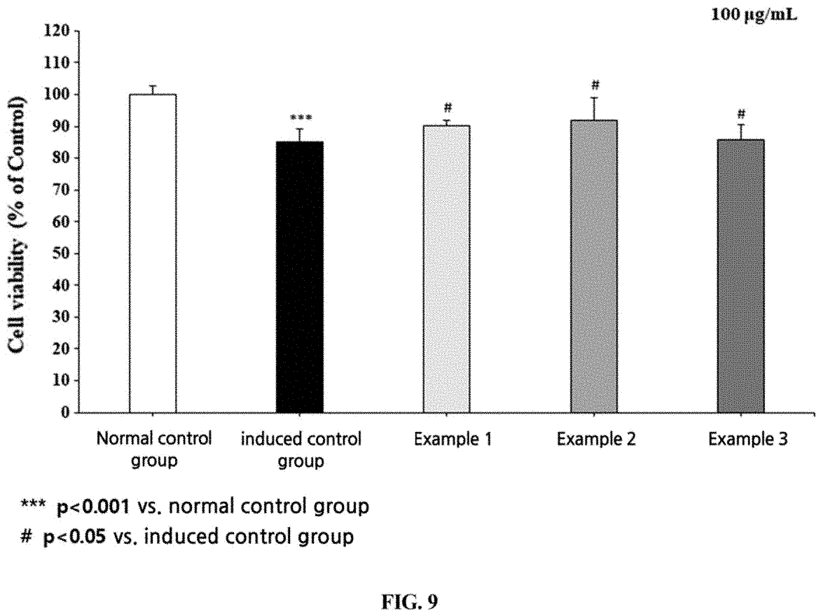

The viability of the human keratinocyte HaCaT cells was measured by culturing the cells at 37° C. for 24 hours in a culture medium in which a sample was diluted to 50 μg/mL or 100 μg/mL and conducting MTT assay. 24 hours after the sample treatment, the culture medium in which the sample was diluted was completely removed and the cells were cultured at 37° C. for 4 hours after treating with 100 μL of a MTT solution diluted with a medium to a concentration of 500 μg/mL. Then, after dissolution by treating with 100% DMSO, absorbance was measured at 540 nm. The result is shown in FIG. 8 and FIG. 9.

As shown in FIG. 8 and FIG. 9, about 15% of UVB-induced cytotoxicity was identified in the induced group as compared to the normal control group, and cell-protecting effect was observed for all of Examples 1, 2 and 3. Among the samples, the gaedarae fruit extract (Example 1) and the gaedarae leaf extract (Comparative Example 3) showed similar cell-protecting effect as compared to the induced group. This means that the gaedarae fruit, leaf and stem extracts of Examples 1, 2 and 3 exhibit cell-protecting effect against UVB irradiation.

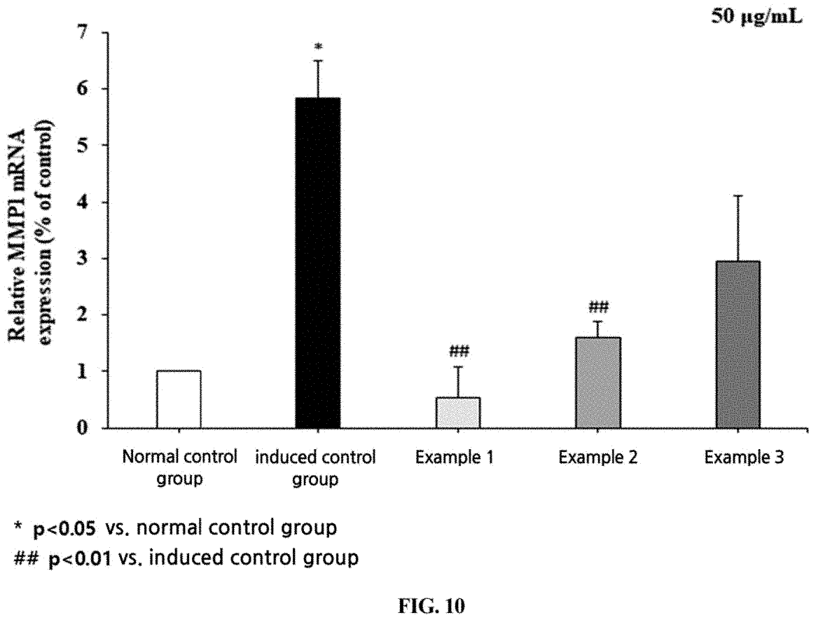

After treating the human keratinocyte HaCaT cells with ultraviolet ray, the cells were treated with Examples 1, 2 and 3 from the different parts of gaedarae (Actinidia polygama) and the MMP1 gene expression level was compared with the normal control group and the induced group. The result is shown in FIG. 10.

As shown in FIG. 10, among the gaedarae extracts of different parts, the gaedarae fruit extract (Example 1) showed the largest decrease by about 90% with respect to the induced group, followed by 72% decrease for the gaedarae leaf extract (Example 2) and 50% for the gaedarae stem extract (Example 3). Accordingly, it was confirmed that the gaedarae fruit extract exhibits the best skin-protecting effect among the gaedarae extracts of different parts.

2. Measurement of Effect of Inhibited Secretion of Interleukin 4 in Rat-Derived RBL-2H3 Mast Cells

Preparation of rat-derived RBL-2H3 mast cells and analysis of IL-4 production were conducted in the same manner as in 4 of Test Example 1.

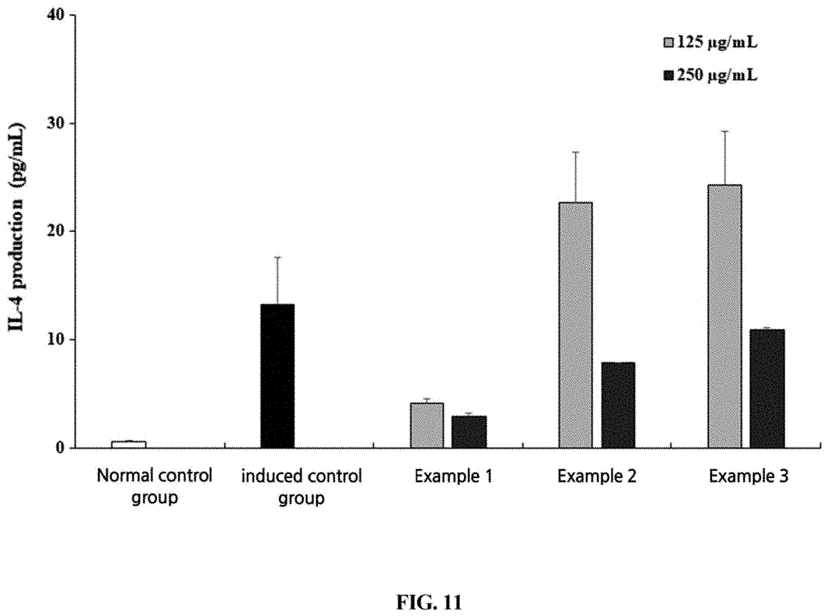

As shown in FIG. 11, the IL-4 secretion amount was 13.2 μg/mL for the induced group, and the secretion amount was decreased as the sample concentration was increased.

IL-4 secretion-inhibiting effect was not observed when the gaedarae leaf extract (Example 2) and the gaedarae stem extract (Example 3) were treated at a final concentration of 125 μg/mL, whereas the gaedarae fruit extract (Example 1) showed an inhibitory effect of 69%.

A very high IL-4 secretion-inhibiting effect of 78% was observed when the gaedarae fruit extract (Example 1) was treated at a final concentration of 250 μg/mL, whereas relatively low inhibitory effect of 40% and 17% was observed for the gaedarae leaf extract (Example 2) and the gaedarae stem extract (Example 3), respectively.

Accordingly, it was confirmed from the comparison of the effect of the gaedarae extracts of different parts in rat-derived mast cells that the gaedarae fruit extract exhibits the most superior effect of inhibiting IL-4 secretion.

### Test Example 3: Test of Ultraviolet Ray-Induced Skin Damage Animal Model

1. Preparation of Experimental Animals and Samples

As a positive control group, “fermented honeybush extract powder” (HU-018, Huons Natural) approved as a second-grade health functional food material helpful in maintaining the health of skin with ultraviolet ray-induced skin damage was used. The fermented honeybush extract powder was a brown powder and the solid content of the fermented honeybush extract powder except an excipient was 50 wt %.

SKH??1 hairless mice (6-week-old, female) acquired from Raon Bio (Yongin, Korea) were used as experimental animals. Solid feed (antibiotic-free) and water were supplied sufficiently until the day of experiment, and the animals were acclimatized for 1 week to an environment of temperature of 23±2° C., humidity of 55±10% and 12-hour light/dark cycles. All the experimental procedures were conducted according to the Principle of Laboratory Animal Care of the NIH (National Institutes of Health) and were approved by the Ethics Committee for Laboratory Animals at Chung-Ang University.

Skin damage was induced by irradiating ultraviolet ray by modifying the method of Im, et al. [Im, A. R., et al., BMC Complementary and Alternative Medicine, 2014. 14(1): p. 424]. The mice were randomly divided into four groups with 8 mice per group and skin damage was induced in three groups among them by irradiating UVB 3 times a week for a total of 6 weeks using BioSpectra (Vilber Lourmat, France), while changing intensity from 50 mJ/cm² (1 MED, minimal erythemal dose) to 70 mJ/cm² with 2-week intervals. Concurrently with the ultraviolet ray irradiation, the gaedarae fruit extract (Example 1, APWE) or the fermented honeybush extract powder (Comparative Example 3, HU-018) was orally administered using a sonde at a dosage of 100 mg/kg/day based on the active ingredient once a day for 6 weeks. Physiological saline was orally administered to a normal control group and an induced group.

TABLE 2

| | |
|---|---|
| Normal control | No ultraviolet ray irradiation, administration |
| group | of physiological saline (Normal) |
| Induced group | Ultraviolet ray irradiation, administration |
| | of physiological saline (UVB + Saline) |
| Example 1 | Ultraviolet ray irradiation, administration |
| | of gaedarae fruit extract (UVB + APWE, 100 |
| | mg/kg/day) |
| Comparative | Ultraviolet ray irradiation, administration |
| Example 3 | of fermented honeybush extract powder (UVB + |
| | HU-018, 100 mg/kg/day) |

2. Measurement of Body Weight

Body weight was measured 4 and 6 weeks after the induction of skin damage. As a result, body weight decrease was observed in all of the induced group, Example 1 and Comparative Example 3 as compared to the normal control group at week 4, although there was no statistical significance. This trend of body weight decrease was observed also at week 6 in all the ultraviolet ray-irradiated groups although there was no statistical significance. It is though that the body weight decrease is due to the ultraviolet ray irradiation.

3. Measurement of Transepidermal Water Loss (TEWL)

Transepidermal water loss (g/m²h) was measured 4, 6 and 8 weeks after the induction of skin damage using Tewameter (Courage Khazaka Electronic GmbH, Cologne, Germany). The measurement was made under a condition of 22-24° C. and 50-60% humidity, and the result was recorded as the mean of TEWL values with smallest deviation, except the initial values.

At week 4, the transepidermal water loss was 15.6 g/m²h for the induced group, whereas Example 1 showed significant decrease as 13.1 g/m²h (p=0.021). Comparative Example 3 also showed significant decrease as compared to the induced group with 14.3 g/m²h although the decrease was smaller than that of Example 1 (p=0.045). At week 6, the transepidermal water loss of the induced group was slightly increased to 16.0 g/m²h as compared to week 4. In contrast, Example 1 and Comparative Example 3 showed significant decrease to 10.8 g/m²h and 13.3 g/m²h, respectively, as compared to the induced group (p=0.0002, p=0.007). In particular, the decrease of transepidermal water loss was about 2 times larger for Example 1 than Comparative Example 3.

TABLE 3

| | Transepidermal water loss (TEWL, g/m² h) | |
|---|---|---|
| | Week 4 | Week 6 |
| Normal control group | 12.8 ± 2.55* | 9.9 ± 1.45*** |
| Induced group | 15.6 ± 0.82 | 16.0 ± 2.16 |
| Example 1 | 13.1 ± 2.60* | 10.8 ± 1.99*** |
| Comparative Example 3 | 14.3 ± 1.45* | 13.3 ± 0.92** |

*p < 0.05;
**p < 0.01;
***p < 0.001

4. Measurement of Skin Moisture Content

Skin moisture content was measured using Corneometer (Courage Khazaka Electronic GmbH, Cologne, Germany) by the same method as the measurement of transepidermal water loss.

At week 4, the skin moisture content was increased for both Example 1 and Comparative Example 3 as compared to the induced group, although there was no statistical significance. At week 6, the skin moisture content of the induced group was decreased to 28.2 A.U. by about 35% with respect to the normal control group (43.5 A.U.). In contrast, Comparative Example 3 and Example 1 showed significant increase to 35.3 A.U. and 38 A.U., respectively (p=0.035, p=0.010).

TABLE 4

Skin moisture content (A.U.)

| | Week 4 | Week 6 |
|---|---|---|
| Normal control group | 40.4 ± 3.58** | 43.5 ± 6.06*** |
| Induced group | 34.1 ± 4.59 | 28.2 ± 6.56 |
| Example 1 | 37.9 ± 6.15 | 35.3 ± 5.49* |
| Comparative Example 3 | 36.6 ± 3.58 | 35.3 ± 5.49* |

*p < 0.05;
**p < 0.01;
***p < 0.001

5. Measurement of Skin Roughness

Visual assessment was conducted at 4, 6 and 8 weeks after the induction of skin damage. The experimental animals were anesthetized with Zoletil and Rompun (0.008 cc/10 g (40 mg/kg)+0.002 cc/10 g (5 mg/kg)) diluted 10-fold in physiological saline, and the skin condition of the anesthetized experimental animals was imaged using a folliscope.

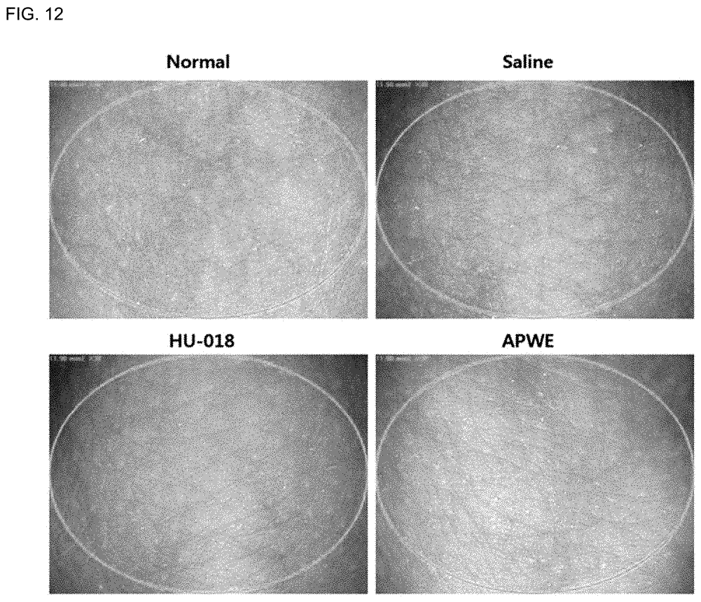

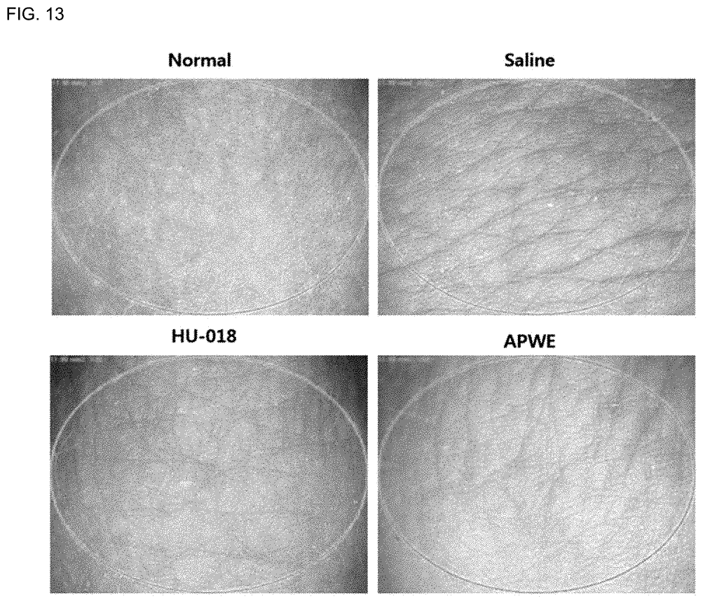

At week 4, the skin roughness was increased in all the ultraviolet ray-irradiated groups as compared to the normal control group. But, the skin roughness was increased in Example 1 and Comparative Example 3 as compared to the induced group. Also, at week 6, distinct improvement in skin roughness was observed for Example 1 and Comparative Example 3 as compared to the induced group (see FIG. 12 and FIG. 13).

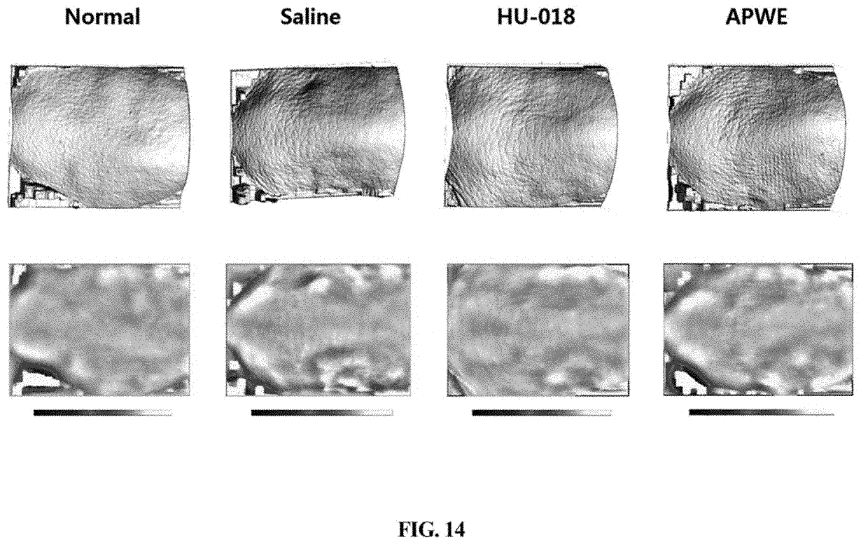

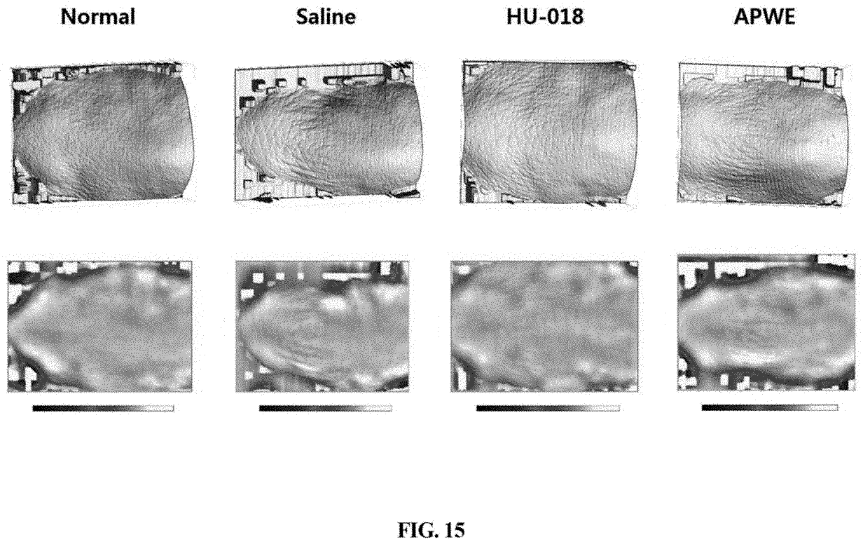

Primos Lite is an instrument for quantitatively and quantitatively analyzing skin microstructure and roughness by refracting a parallel fringe with a slight difference in height on skin surface. At weeks 4 and 6 after the induction of skin damage, 3D images and skin roughness images were obtained using the 3D system. The result is shown in FIG. 14 and FIG. 15. In addition, Rₐ (average skin roughness), Rₘₐₓ (maximum skin roughness: the largest difference in skin height of evenly divided 5 zones) and Rₜ (maximum skin roughness: the difference of the highest and lowest skin surface) were measured, and the result is shown in Table 5, Table 6 and Table 7, respectively.

At week 4 after the induction of ultraviolet ray-induced skin damage, there was no statistically significant difference in Rₐ among the test groups. But, at week 6, statistically significant decrease in Rₐ was observed for Example 1 as compared to the induced group (p=0.010) (see Table 5). As a result of monitoring Rₘₐₓ and Rₜ as other indices of skin roughness, no significant difference was observed among the test groups at week 4. But, at week 6, statistically significant decrease in Rₘₐₓ and Rₜ values was observed for Example 1, like the Rₐ value, as compared to the induced group (p=0.016, p=0.009) (see Table 6 and Table 7). No significant decrease in skin roughness was observed for Comparative Example 3 in terms of the Rₐ, Rₘₐₓ and Rₜ values.

TABLE 5

Rₐ

| | Week 4 | Week 6 |
|---|---|---|
| Normal control group | 22.69 ± 1.42 | 21.84 ± 1.21* |
| Induced group | 23.42 ± 3.34 | 24.96 ± 3.01 |
| Example 1 | 24.45 ± 2.49 | 21.54 ± 1.21* |
| Comparative Example 3 | 23.69 ± 2.93 | 23.77 ± 2.27 |

*p < 0.05;
** p < 0.01;
*** p < 0.001

TABLE 6

Rₘₐₓ

| | Week 4 | Week 6 |
|---|---|---|
| Normal control group | 152.95 ± 10.12 | 156.12 ± 10.69* |
| Induced group | 167.13 ± 27.23 | 180.23 ± 25.52 |
| Example 1 | 173.54 ± 22.02 | 154.16 ± 8.64* |
| Comparative Example 3 | 167.40 ± 23.86 | 169.06 ± 24.92 |

*p < 0.05;
** p < 0.01;
*** p < 0.001

TABLE 7

Rₜ

| | Week 4 | Week 6 |
|---|---|---|
| Normal control group | 165.13 ± 11.63 | 166.04 ± 11.12* |
| Induced group | 177.46 ± 26.98 | 192.34 ± 26.53 |
| Example 1 | 184.15 ± 123.49 | 161.88 ± 9.77** |
| Comparative Example 3 | 177.54 ± 24.08 | 177.51 ± 26.52 |

*p < 0.05;
**p < 0.01;
*** p < 0.001

6. Measurement of Skin Elasticity

Skin elasticity was measured 4 and 8 weeks after the induction of ultraviolet ray-induced skin damage using two types of instruments (Cutometer and Ballistometer). Cutometer dual MPA 580 (Courage and Khazaka Electronic GmbH, Cologne, Germany) measures the elasticity of the dermal layer based on the suction method, whereby the skin drawn into a probe by a negative pressure is restored to the original state after the negative pressure is removed. R (R0-R9), F (F1-F4) and Q (Q0-Q3) parameters are measured.

Ballistometer (Dia-Stron Ltd., Andover, UK) is an instrument for measuring the elasticity, resilience, firmness, softness, swelling, etc. of a narrow or uneven skin part, which is difficult to analyze with the conventional instruments, by applying vibrational energy and analyzing waveforms.

As a result of measuring R7, which is indicative of skin firmness, using Cutometer MPA 580, no statistically significant difference was observed among the test groups at week 4. However, at week 6, significant increase of R7 by about 43% was observed for Example 1 as compared to the induced group (p=0.041) (see Table 8).

Meanwhile, as a result of measuring alpha value, as a parameter for skin elasticity, using Ballistometer, no statistically significant difference was observed among the test groups except the normal control group 4 weeks after the ultraviolet ray irradiation. At week 6, both Example 1 and Comparative Example 3 showed statistically significant difference of the alpha value as compared to the induced group (p=0.013, p=0.001) (see Table 9).

TABLE 8

Skin firmness (Cutometer R7)

| | Week 4 | Week 6 |
|---|---|---|
| Normal control group | 0.095 ± 0.058 | 0.101 ± 0.011 |
| Induced group | 0.105 ± 0.020 | 0.075 ± 0.039 |
| Example 1 | 0.110 ± 0.021 | 0.107 ± 0.011* |
| Comparative Example 3 | 0.089 ± 0.050 | 0.097 ± 0.009 |

*p < 0.05;
** p < 0.01;
*** p < 0.001

TABLE 9

| | Skin elasticity (Ballistometer alpha) | |
|---|---|---|
| | Week 4 | Week 6 |
| Normal control group | 0.020 ± 0.005* | 0.017 ± 0.002*** |
| Induced group | 0.036 ± 0.008 | 0.041 ± 0.003 |
| Example 1 | 0.034 ± 0.010 | 0.033 ± 0.004** |
| Comparative Example 3 | 0.037 ± 0.004 | 0.034 ± 0.005* |

*p < 0.05;
**p < 0.01;
***p < 0.001

### Test Example 4: Comparison of Administration Routes of Gaedarae (Actinidia polygama)

1. Preparation of Experimental Animals and Samples

Preparation of experimental animals and induction of skin damage by ultraviolet ray irradiation were conducted in the same manner as in Test Example 3.

Concurrently with the ultraviolet ray irradiation, the gaedarae fruit extract (Example 1, APWE) was orally administered using a sonde at a dosage of 100 mg/kg/day based on the active ingredient or the same dosage was applied on the ultraviolet ray-irradiated skin (Comparative Example 4), once a day for 6 weeks. Physiological saline was orally administered to a normal control group and an induced group.

TABLE 10

| | |
|---|---|
| Normal control | No ultraviolet ray irradiation, oral administration |
| group | of physiological saline (Normal) |
| Induced group | Ultraviolet ray irradiation, oral administration |
| | of physiological saline (UVB + Saline) |
| Example 1 | Ultraviolet ray irradiation, oral administration of |
| | gaedarae fruit extract (UVB + APWE, 100 mg/kg/day) |
| Comparative | Ultraviolet ray irradiation, transdermal administration |
| Example 4 | of gaedarae fruit extract (UVB + APWE, 100 mg/kg/day) |

2. Measurement of Transepidermal Water Loss (TEWL)

Transepidermal water loss (g/m²h) was measured 6 weeks after the induction of skin damage using Tewameter (Courage Khazaka Electronic GmbH, Cologne, Germany). The measurement was made in the same manner as in Test Example 3, and the relative ratio of the transepidermal water loss of the normal control group, Example 1 and Comparative Example 4 at week 6 with respect to that of the induced group as 100% is shown in Table 11.

The oral administration of the gaedarae fruit extract (Example 1) resulted in significant decrease of transepidermal water loss to 67.5% as compared to the induced group (p=0.000212). The transdermal administration of the gaedarae fruit extract (Comparative Example 4) resulted in slight decrease of transepidermal water loss to about 88% as compared to the induced group, but there was no significant decrease from the induced group (p=0.264926).

TABLE 11

| | Transepidermal water loss (%) | p-value |
|---|---|---|
| Normal control group | 61.796888 ± 9.090551*** | 0.000012 |
| Induced group | 100 ± 16.3355 | |
| Example 1 | 67.5 ± 12.46871*** | 0.000212 |
| Comparative Example 4 | 88.03738 ± 1.218543 | 0.264926 |

***p < 0.001

3. Measurement of Skin Moisture Content

Also, skin moisture content was measured 6 weeks after the induction of skin damage using Corneometer (Courage Khazaka Electronic GmbH, Cologne, Germany) by the same method as the measurement of transepidermal water loss. The relative ratio of the skin moisture content of the normal control group, Example 1 and Comparative Example 4 at week 6 with respect to that of the induced group as 100% is shown in Table 12.

The oral administration of the gaedarae fruit extract (Example 1) resulted in remarkable increase of skin moisture content to about 134% as compared to the induced group (p=0.000261). The transdermal administration of the gaedarae fruit extract (Comparative Example 4) resulted in insignificant increase of skin moisture content to about 104% as compared to the induced group (p=0.240736).

TABLE 12

| | Skin moisture content (%) | p-value |
|---|---|---|
| Normal control group | 154.211 ± 21.4987*** | 0.000261 |
| Induced group | 100 ± 18.09641 | |
| Example 1 | 134.7518 ± 23.63872* | 0.010252 |
| Comparative Example 4 | 103.8283 ± 2.943829 | 0.240736 |

*p < 0.05;
***p < 0.001

4. Measurement of Skin Elasticity

Skin elasticity was measured 6 weeks after the induction of skin damage using Ballistometer. The relative ratio of the skin elasticity (Ballistometer alpha value) of the normal control group, Example 1 and Comparative Example 4 at week 6 with respect to that of the induced group as 100% is shown in Table 13.

The oral administration of the gaedarae fruit extract (Example 1) resulted in remarkable decrease of skin elasticity to about 81% as compared to the induced group (p=0.000938). The transdermal administration of the gaedarae fruit extract (Comparative Example 4) resulted in insignificant change of skin elasticity to about 102% as compared to the induced group (p=0.941682).

TABLE 13

| | Skin elasticity | |
|---|---|---|
| | (Ballistometer alpha) (%) | p-value |
| Normal control group | 41.46341 ± 5.830383*** | 0.00000000013 |
| Induced group | 100 ± 11.03063 | |
| Example 1 | 80.79268 ± 9.350225*** | 0.000938 |
| Comparative Example 4 | 102.1429 ± 13.96972 | 0.941682 |

***p < 0.001

Statistical Analysis

The experimental data were presented as mean±standard error of the mean (S.E.M). Significance was tested by one way analysis of variance (ANOVA) and Turkey's HDS method was used for post-hoc testing among groups. P<0.05 was assumed to be statistically significant.

Hereinafter, formulation examples of a composition containing the extract of the present disclosure are described. However, they are intended only to illustrate, not to limit, the present disclosure.

### Formulation Example 1: Preparation of Powder

| | | |
|---|---|---|
| Gaedarae fruit extract powder of Preparation Example | 20 | mg |
| Lactose | 100 | mg |
| Talc | 10 | mg |

A powder was prepared by mixing the above ingredients and filling in an airtight pouch.

### Formulation Example 2: Preparation of Tablet

| | | |
|---|---|---|
| Gaedarae fruit extract powder of Preparation Example | 10 | mg |
| Cornstarch | 100 | mg |
| Lactose | 100 | mg |
| Magnesium stearate | 2 | mg |

A tablet was prepared according to a common tablet-making method after mixing the above ingredients.

### Formulation Example 3: Preparation of Capsule

| | | |
|---|---|---|
| Gaedarae fruit extract powder of Preparation Example | 10 | mg |
| Crystalline cellulose | 3 | mg |
| Lactose 1 | 4.8 | mg |
| Magnesium stearate | 0.2 | mg |

A capsule was prepared according to a common method by mixing the above ingredients and filling in a gelatin capsule.

### Formulation Example 4: Preparation of Granule

| Gaedarae fruit extract powder of Preparation Example | 1,000 | mg |
|---|---|---|
| Vitamin mixture | Adequate | |
| Vitamin A acetate | 70 | μg |
| Vitamin E | 1.0 | mg |
| Vitamin B₁ | 0.13 | mg |
| Vitamin B₂ | 0.15 | mg |
| Vitamin B₆ | 0.5 | mg |
| Vitamin B₁₂ | 0.2 | μg |
| Vitamin C | 10 | mg |
| Biotin | 10 | μg |
| Nicotinamide | 1.7 | mg |
| Folic acid | 50 | μg |
| Calcium pantothenate | 0.5 | mg |
| Mineral mixture | Adequate | |
| Ferrous sulfate | 1.75 | mg |
| Zinc oxide | 0.82 | mg |
| Magnesium carbonate | 25.3 | mg |
| Monopotassium phosphate | 15 | mg |
| Dicalcium phosphate | 55 | mg |
| Potassium citrate | 90 | mg |
| Calcium carbonate | 100 | mg |
| Magnesium chloride | 24.8 | mg |

Although the above-described compositions of vitamin and mineral mixtures are presented as specific examples suitable for health functional foods, they may be changed as desired. After preparing a granule by mixing the above ingredients, a health functional food composition was prepared according to a common health functional food preparation method.

### Formulation Example 5: Preparation of Beverage

| | | |
|---|---|---|
| Gaedarae fruit extract powder of Preparation Example | 1,000 | mg |
| Citric acid | 1,000 | mg |
| Oligosaccharide | 100 | g |
| Plum concentrate | 2 | g |
| Taurine | 1 | g |
| Purified water | To 900 | mL |

After mixing the above ingredients, the mixture was heated at 85° C. for about 1 hour with stirring. The prepared solution was filtered, collected in a sterilized 2-L container, sealed, sterilized and then stored in a refrigerator until use for preparation of a functional beverage composition of the present disclosure.

### Formulation Example 6: Preparation of Animal Feed Composition

A feed composition for animals (pets) was prepared by mixing 0.1 kg of the gaedarae fruit extract powder of Preparation Example, 25.5 kg of corn, 15.04 kg of wheat, 8.15 kg of wheat flour, 7.4 kg of rice bran, 18 kg of soybean meal, 1 kg of corn gluten meal, 14 kg of chicken meal, 9 kg of animal fat, 0.3 kg of processed salt, 0.3 kg of tricalcium phosphate, 1 kg of limestone, 0.01 kg of choline chloride, 0.05 kg of vitamins, 0.05 kg of minerals and 0.1 kg of digestive enzymes.

### Formulation Example 7: Preparation of Nourishing Toilet Water

| Gaedarae fruit extract powder of Preparation Example | 0.05 | wt % |
|---|---|---|
| Vaseline | 2.0 | wt % |
| Sorbitan sesquioleate | 0.8 | wt % |
| Polyoxyethylene oleyl ether | 1.2 | wt % |
| Methyl p-oxybenzoate | Adequate | |
| Propylene glycol | 5.0 | wt % |
| Ethanol | 3.2 | wt % |
| Carboxyvinyl polymer | 18.0 | wt % |
| Potassium hydroxide | 0.1 | wt % |

| | |
|---|---|
| Pigment | Adequate |
| Flavor | Adequate |

A nourishing toilet water was prepared according to a common method by mixing the above ingredients.

### Formulation Example 8: Preparation of Mask Pack

| | | |
|---|---|---|
| Gaedarae fruit extract powder of Preparation Example | 0.05 | wt % |
| Dlycerin | 5.0 | wt % |
| Propylene glycol | 4.0 | wt % |
| Polyvinyl alcohol | 15.0 | wt % |
| Ethanol | 8.0 | wt % |
| Polyoxyethylene oleyl ether | 1.0 | wt % |
| Methyl p-oxybenzoate | 0.2 | wt % |

| | |
|---|---|
| Pigment | Adequate |
| Flavor | Adequate |

A mask pack was prepared according to a common method by mixing the above ingredients.

### Formulation Example 9: Preparation of Essence

| | | |
|---|---|---|
| Gaedarae fruit extract powder of Preparation Example | 0.2 | wt % |
| Propylene glycol | 10.0 | wt % |
| Glycerin | 10.0 | wt % |
| Aqueous sodium hyaluronate solution (1%) | 5.0 | wt % |
| Ethanol | 5.0 | wt % |
| Polyoxyethylene hydrogenated castor oil | 1.0 | wt % |
| Methyl p-oxybenzoate | 0.1 | wt % |

| | |
|---|---|
| Flavor | Adequate |
| Purified water | Balance |

An essence was prepared according to a common method by mixing the above ingredients.

We claim:

1.-11. (canceled)

12. A method for alleviating or treating ultraviolet ray-induced skin damage or moisturizing skin in a subject in need thereof, comprising a step of administering a food composition or a pharmaceutical composition comprising a gaedarae (Actinidia polygama) extract as an active ingredient, and carrier, excipient or diluent.

13. The method according to claim 12, wherein the ultraviolet ray-induced skin damage is skin dryness, reduced skin elasticity or skin roughness.

14. The method according to claim 12, wherein the gaedarae (Actinidia polygama) extract is an extract of gaedarae fruit.

15. The method according to claim 12, wherein the gaedarae (Actinidia polygama) extract is an extract obtained with water, a C1-4 alcohol or a mixture solvent thereof.

16. The method according to claim 15, wherein the food composition is formulated into a powder, a granule, a tablet, a capsule, a pill, an extract, a jelly, a tea bag or a beverage.

17. The method according to claim 12, wherein the food composition is a health functional food for alleviating ultraviolet ray-induced skin damage.

18. The method according to claim 12, wherein the food composition is a health functional food for moisturizing skin.

19. The method according to claim 12, wherein the food composition or pharmaceutical composition further comprising one or more selected from a group of fermented honeybush extract powder; pine bark extract; red ginseng, torilis fructus, and corni fructus complex extract; fingerroot extract powder; probiotics; konjac potato extract; rice bran extract; lithospermi radix extract powder; collagen peptide; dandelion complex extract; corn germ extract; fermented bean and barley mixture; wheat germ oil extract; pomegranate concentrate; N-acetylglucosamine; spirulina; chloroplast-containing plant; chlorella; phosphatidylserine; hyaluronic acid; and aloe gel.
